(19) United States
(12) Patent Application Publication
(10) Pub. No.: US 2014/0356468 A1
(43) Pub. Date: Dec. 4, 2014

(54) COMPOSITION CONTAINING PAPER MULBERRY EXTRACTS

(71) Applicant: AMOREPACIFIC CORPORATION, Seoul (KR)

(72) Inventors: Jin Young LEE, Yongin-si (KR); Hyang Tae CHOI, Yongin-si (KR); Han Byul KIM, Yongin-si (KR); Ji Seong KIM, Yongin-si (KR)

(73) Assignee: PACIFIC CORPORATION, Seoul (KR)

(21) Appl. No.: 14/464,044

(22) Filed: Aug. 20, 2014

Related U.S. Application Data
(63) Continuation of application No. 13/808,021, filed on Jan. 2, 2013.

(30) Foreign Application Priority Data
Jul. 2, 2010 (KR) 10-2010-0063736
Jul. 2, 2010 (KR) 10-2010-0063879
Jul. 2, 2010 (KR) 10-2010-0063990
Jul. 2, 2010 (KR) 10-20100063878
Jul. 5, 2010 (KR) 10-2010-0064296
Jul. 5, 2010 (KR) 10-2010-0064367
Jul. 13, 2010 (KR) 10-2010-0067463

(51) Int. Cl. A61K 36/605

(52) U.S. Cl. CPC A61K 36/605
USPC 424/769

(57) ABSTRACT

Disclosed is a use of a composition containing a paper mulberry extract, and more particularly to a use of a composition containing, as an active ingredient, a paper mulberry extract in enhancement of skin moisturization, inhibition of skin aging, alleviation of inflammation, antibacterial activity, pore size reduction, sebum control, skin complexion improvement, decomposition of subcutaneous fat, stimulation of melanin synthesis, and gray hair prevention.

### CROSS-REFERENCE TO RELATED APPLICATIONS

This is a Continuation of U.S. application Ser. No. 13/808,021 filed Jan. 2, 2013 which is a National Stage Entry of PCT International Application No. PCT/KR2011/004890 filed Jul. 4, 2011, which claims benefit of Korean Patent Application Nos. 10-2010-0067463 filed Jul. 13, 2010, 10-2010-0064296 filed Jul. 5, 2010, 10-2010-0064367 filed Jul. 5, 2010, 10-2010-0063878 filed Jul. 2, 2010, 10-2010-0063879 filed Jul. 2, 2010, 10-2010-0063990 filed Jul. 2, 2010, and 10-2010-0063736 filed Jul. 2, 2010, of which disclosures are incorporated herein by reference in their entirety.

### TECHNICAL FIELD

The present invention relates to a composition containing a paper mulberry extract, and more particularly, to a composition which can various effects on the skin.

### BACKGROUND ART

The skin is the primary barrier of the human body, which functions to protect the organs of the body from external environmental stimuli such as changes in temperature and humidity, UV rays and pollutants. However, the function of the skin is reduced due to intrinsic and extrinsic factors. With respect to the intrinsic factors, the secretion of various hormones that regulate metabolisms decreases with aging, and the function of immune cells and the activity of cells decrease with aging, so that the biosynthesis of immune proteins need in the body and constituent proteins of the body decreases. With respect to the extrinsic factors, due to physical and chemical stimuli and stresses caused by increases in exposure to UV rays, free radicals and reactive oxygen species resulting from environmental pollution such as ozone layer destruction, various changes in the skin occur, including reduced skin function, stimulated skin aging, bad complexion, and darker skin tone.

These days, because most people want to look young and beautiful, they have a strong need to prevent or ameliorate or resolve the changes in the skin resulting from the intrinsic and extrinsic factors. Thus, wrinkle-reducing agents and elasticity-improving agents and the like have been developed.

Meanwhile, skin conditions, including wrinkles, loss of elasticity, xerosis (dry skin), etc., result from changes or decreases in the contents and arrangements of collagen, elastin, hyaluronic acid, proteoglycan, glycosaminogluycan, fibronectin and glycoprotein in the skin, and a decrease in the ability to bind to water, which result in a decrease in the matrix of the skin and the loss of the moisturizing ability of the skin. In addition, it is known that, in most of cells constituting the skin, the biosynthesis of cyclooxygenase-2 (Cox-2) that produces proinflammatory cytokines, including tumor necrosis factor-α (TNFα), interleukin-1β (IL-1β) and prostaglandin, increases, and the biosynthesis of matrix metalloproteinase (MMP) that degrades skin tissue is increased by these inflammatory factors.

Thus, the development of substances capable of making the skin thicker by proliferating skin cells or increasing the matrix of the skin, substances capable of inhibiting the biosynthesis of cyclooxygenase-2 (Cox-2), substances capable of inhibiting the biosynthesis of tumor necrosis factors, and substances that stimulate the production of tropoelastin and fibrillin in a fibroblast, and the like, makes it possible to ameliorate various skin conditions, including wrinkles, elasticity loss and xerosis.

### DISCLOSURE

### Technical Problem

Accordingly, the present inventors have conducted studies to find natural substances capable of improving various skin conditions, and as a result, have found that a paper mulberry extract can improve various skin conditions related to skin moisturization, skin elasticity and the like and has the effects of decomposing subcutaneous fat and preventing gray hair, thereby completing the present invention.

Therefore, it is an object of the present invention to provide a composition containing a natural substance which can improve general skin conditions, has an excellent effect of decomposing fat and can prevent gray hair.

### Technical Solution

In order to accomplish the above object, the present invention provides a composition containing a paper mulberry extract.

The present invention also provides the use of a composition containing a paper mulberry extract for skin moisturization, anti-aging, anti-inflammation, slimming and gray hair prevention.

### Advantageous Effects

The composition of the present invention contains a paper mulberry extract which has skin moisturization, anti-aging, antioxidant and anti-inflammatory effects, and thus can improve general skin conditions. In addition, the composition of the present invention has the effects of reducing pore size, controlling sebum, alleviating acne conditions and improving complexion. Further, the composition of the present invention can prevent gray hair by stimulating melanin synthesis and make the skin elastic and smooth by reducing body fat mass.

### BEST MODE

The composition of the present invention contains a paper mulberry extract as an active ingredient.

Plants belonging to the genus Broussonetia (paper mulberry) which is used as an active ingredient in the present invention include Broussonetia kazinoki Sieb, Broussonetia papyrifera Vent, Broussonetia kazinoki var. humilis and the like. These plants are deciduous broad-leaf shrubs that are distributed in most areas of Korea (mainly the southern part), China, Taiwan, Japan and the like and grow in sunny places of mountains, places around fields, etc. The bast fiber of paper mulberry has been used as a raw material for making paper, and it is known that paper mulberry has various medicinal effects, including tonic, eyesight improvement, impotence alleviation, dropsy treatment, “Gi”, palsy removal, augmentation, blood clarification, and the like.

The definition of the paper mulberry extract that is used in the present invention includes not only an extract obtained by extracting paper mulberry, but also a concentrate obtained by concentrating some or all of the extract, and an infusion, decoction, tincture and fluid extract obtained after drying the concentrate, as well as active ingredients contained in the paper mulberry, and also the plant itself. In addition, the extract that is used in the present invention may be extracts obtained from all the portions (including stem, root, leaf, flower, fruit, etc.) of paper mulberry and is not limited to an extract of any particular portion of paper mulberry.

The paper mulberry extract that is used in the present invention can be prepared according to any method known in the art. For example, paper mulberry is dried by any method such as natural drying or forced drying and cut finely, after which it is extracted by any method such as cold maceration, percolation or warm maceration using a polar solvent such as water, ethanol, butanol or acetone, or a non-polar solvent such as ether, hexane, benzene, chloroform or ethyl acetate, or a mixed solvent of the non-polar solvent and the polar solvent, or a solvent such as alkaline water, or vegetable oil such as bean oil or sesame oil, thereby obtaining an extract containing an active ingredient. With the extraction process, cold maceration and percolation are preferably carried out for 12-96 hours, and warm maceration is preferably carried out at a temperature close to the reflux temperature of solvent for 0.5-24 hours depending on the kind of solvent used and the temperature of maceration. Particularly, a tincture, extract or liquid extract obtained by extraction in hydrated alcohol is preferably used.

The cosmetic composition according to the present invention may contain the paper mulberry extract in an amount of 0.0001-90 wt %, for example, 0.1-70 wt %, preferably 1-50 wt %, and more preferably 1-20 wt %, based on the total weight of the composition. When the content of the paper mulberry extract in the composition is within the above range, the effect of improving skin conditions can be obtained while concerns about skin safety and formulation stability will not occur.

The composition according to the present invention can be used as a skin-moisturizing composition. The skin-moisturizing composition can be used to enhance skin barrier function and induce skin keratinocyte differentiation. Thus, the composition of the present invention can prevent or ameliorate xerosis, atopic dermatitis, contact dermatitis or psoriasis, which results from imperfect epidermal differentiation.

In addition, the composition according to the present invention can be used as an anti-aging composition. The anti-aging composition can inhibit the expression of collagenase to increase skin elasticity and reduce wrinkles.

In addition, the composition according to the present invention can be used as an antimicrobial and anti-inflammatory composition. The antimicrobial and anti-inflammatory composition has a very excellent antimicrobial effect, particularly against acne-causing microorganisms, and also has an effect of reducing the expression of inflammatory factors to provide anti-inflammatory effects. Thus, it can be used to inhibit skin trouble and alleviate acne.

In addition, the composition according to the present invention can be used as a composition for reducing pore size and controlling sebum. The composition for reducing pore size and controlling sebum serves to stimulate collagen synthesis to reduce pore size and inhibit excessive secretion of sebum. Moreover, the composition has an excellent antioxidant activity of removing reactive oxygen species, and thus can protect the skin from stimuli.

In addition, the composition according to the present invention can be used as a composition for improving complexion and skin tone. When the composition is applied to the skin, it enlarges capillary vessels and promotes blood circulation to enable smooth supply of nutrients to the skin and inhibits skin aging to improve complexion and skin tone.

In addition, the composition according to the present invention can be used as a slimming composition. The slimming composition is effective in decomposing triglyceride and reducing cellulite to make the figure slim. Thus, when the composition is applied to the skin, it exhibits a very excellent slimming effect of decomposing subcutaneous fat.

Additionally, the composition according to the present invention can be used as a composition for preventing gray hair and treating leukoplakia.

It has been proposed that gray hair is caused by the loss of melanocyte stem cells and a reduction in the activity of melanocytes. Particularly, it is known that gray hair due to aging is caused mainly by the loss of stem cells and the occurrence of gray hairs including premature gray hair is attributable to the reduction in activity of melanocytes by environmental and mental stress.

The activity of melanocytes to synthesize melanin is greatly influenced by the activity of MITF, the paper mulberry extract-containing composition according to the present invention can significantly increase the expression of MITF in melanocytes, thereby inhibiting gray hair and stimulating the induction of black hair.

The composition of the present invention can be used as a skin external composition and can be prepared as a cosmetic composition or a pharmaceutical composition.

The skin external composition according to the present invention may be formulated containing a cosmetically and dermatologically acceptable medium or base. The composition may be formulated as a preparation for topical application. Examples of formulations for topical application include solution, gel, solid or dough anhydride, emulsion prepared by dispersing oil phase in a water phase, suspension, microemulsion, microcapsule, microgranule, ionic (liposome) and/or non-ionic vesicle, cream, skin, lotion, powder, ointment, spray, pack, skin adhesive and conceal stick. Also, the skin external composition according to the present invention can be formulated as a foam composition or an aerosol composition further containing a compressed propellant. In addition, the composition according to the present invention can be prepared according to a conventional method known in the art.

The cosmetic composition according to the present invention may contain additives which are conventionally used in the cosmetic field or the skin science field, for example, fatty substance, organic solvent, resolvent, thickener, gelling agent, softener, antioxidant, suspending agent, stabilizer, foaming agent, aromatic, surfactant, water, ionic or non-ionic emulsifying agent, filler, sequestering agent, chelating agent, preservative, vitamins, blocker, wetting agent, essential oil, dye, pigment, hydrophilic or hydrophobic activator, lipid vesicle or other conventional components. These additives are contained in amounts which are generally used in the cosmetic field or the skin science field.

When the composition of the present invention is used for medicine, it may be prepared as solid, semisolid or liquid parenteral administration forms (for transdermal administration or external application) by adding a commonly used inorganic or organic carrier to the active ingredient paper mulberry extract. Examples of formulations for parenteral administration include ointment, lotion, spray, suspension, etc. The composition of the present invention may be formulated according a conventional method known in the art. For formulation, a surfactant, an excipient, a colorant, a flavor, a preservative, a stabilizer, a buffer, a suspension, or other conventional additives may be used adequately.

The dose of the composition according to the present invention may vary depending on the age, sex and body weight of the subject in need of treatment, the particular disease or condition to be treated, the severity of the disease or condition, administration route, or the prescriber's decision. The determination of the administration dose considering these factors will be easily understood by those skilled in the art. The composition may be externally applied to the area to be treated. The administration dose of the composition may generally be from about 0.001 to about 2,000 mg/kg/day. More specifically, the administration dose may be from 200 μg/kg/day to about 5 mg/kg/day.

In addition, the composition for preventing gray hair and treating leukoplakia according to the present invention can be easily formulated as shampoo, rinse, conditioner, tonic or scalp essence which is to be applied to hair or scalp. The composition according to the present invention may contain additives which are conventionally used in the cosmetic field, for example, fatty substance, organic solvent, resolvent, thickener, gelling agent, softener, antioxidant, suspending agent, stabilizer, preservative, vitamins, blocker, wetting agent, essential oil, dye, pigment, hydrophilic or hydrophobic activator, lipid vesicle or other conventional components. These additives are contained in amounts which are generally used in the cosmetic field.

In addition, the skin external composition according to the present invention may comprise, in addition to the paper mulberry extract as an essential ingredient, components capable of exhibiting synergistic effects with the paper mulberry extract. These other components can be suitably selected by those skilled in the art depending on the intended use of the formulation. Additionally, the composition of the present invention may further comprise a substance of promoting absorption into the skin in order to increase the effects thereof.

### MODE FOR INVENTION

Hereinafter, the present invention will be described in further detail with reference to examples and test examples. It is to be understood, however, that these examples are for illustrative purposes only and are not intended to limit the scope of the present invention.

### Preparation Example 1

### Preparation of Paper Mulberry Extract

1 kg of the dried whole plant of paper mulberry was added to 10 L of purified water and heated until boiling, after which it was additionally heated for 10 minutes. Then, the water was removed, and the residue was washed and then washed by addition of 10 L of purified water. The residue was air-dried, added to 20 L of 70% ethanol, connected to a reflux system, heated, and extracted under reflux for 24 hours. The extract was filtered through a 80-mesh sieve to remove the solid, and the remaining filtrate was filtered again and concentrated using a vacuum evaporator to remove the solvent, thereby obtaining about 50 g of green solid powder.

### Preparation Example 2

### Preparation of Paper Mulberry Extract

1 kg of the stem, root, leaf, flower and fruit of paper mulberry were added to 10 L of clean water and extracted by boiling in an extractor equipped with a cooling condenser for 5 hours. The extract was filtered through a 300-mesh filter cloth and allowed to stand at 5 to 15° C. for 5 days, after which it was filtered through filter paper. The filtrate was concentrated under reduced pressure in a distillation device system with a cooling condenser, thereby obtaining 70 g of an extract of the flower and fruit of paper mulberry.

### Test Example 1

### Measurement of Effect on Stimulation of Keratinocyte Differentiation

In order to examine the effect of the paper mulberry extract, obtained in Preparation Example 1, on the stimulation of keratinocyte differentiation, the amount of cornified envelop (CE) produced during keratinocyte differentiation was measured based on absorbance in the following manner.

First, human keratinocytes obtained by primarily culturing cells separated from the epidermis of neonates were placed in a culture flask and attached to the bottom, after which the cell culture was treated with 5 ppm of each of test materials shown in Table 1 below, and then the cells were cultured to a confluence of about 70-80% for 5 days. Herein, the low calcium (0.03 mM)-treated group and the high calcium (1.2 mM)-treated group were used as a negative control group and a positive control group, respectively. Then, the cultured cells were harvested and washed with PBS (phosphate buffered saline), and then 1 ml of Tris-HCl (pH 7.4) containing 2% SDS (sodium dodecyl sulfate) and 20 mM DTT (dithiothreitol) was added to the cells. Then, the cells were sonicated, boiled and centrifuged, and the precipitate was suspended in 1 ml of PBS, and the absorbance at 340 was measured. Meanwhile, a portion of the solution following the sonication was taken and the protein content of the taken portion was measured and used as a standard for the evaluation of cell differentiation. The results of the measurement are shown in Table 1 below.

TABLE 1

| Test material | Keratinocyte differentiation (%) |
|---|---|
| Low calcium (0.03 mM) solution | 100 |
| (negative control) | |
| High calcium (1.2 mM) solution | 210 |
| (positive control) | |
| Paper mulberry extract | 140 |
| (Preparation Example 1) | |

As can be seen in Table 1 above, treatment with the paper mulberry extract of Preparation Example 1 showed an excellent effect on the stimulation of keratinocyte differentiation.

### Test Example 2

### Measurement of Effect on Restoration of Skin Barrier Function

In order to measure the effect of the paper mulberry extract on the restoration of the skin barrier function impaired due to skin damage, the following test was performed. The skin barrier of the upper arm of each of 10 adult men and women was impaired using a tape stripping method, and each of the compositions of Example 1 and Comparative Example 1 shown in Table 2 below was applied to the impaired portion while the degree of restoration of transepidermal water loss (TEWL) was measured using Vapometer (Delfin, Finland) once a day for 7 days. Herein, Example 1 is a composition containing the extract of Preparation Example 1, and Comparative Example 1 is a vehicle as a negative control. The results of the measurement are shown in Table 3 below. The results in Table 3 are expressed as percentages of values after treatment relative to 100% before treatment.

TABLE 2

| Component | Example 1 | Comparative Example 1 |
|---|---|---|
| Purified water | 69 | 70 |
| Propylene glycol | 30 | 30 |
| Paper mulberry extract | 1 | — |
| (Preparation Example 1) | | |

TABLE 3

| | Change (%) in TEWL | | | | | | |
|---|---|---|---|---|---|---|---|
| | Before | 1 | 2 | 3 | 4 | 5 | 6 |
| Test group | treatment | days | days | days | days | days | days |
| Example 1 | 100 | 87 | 72 | 53 | 29 | 12 | 11 |
| Comparative | 100 | 121 | 112 | 98 | 70 | 62 | 43 |
| Example 1 | | | | | | | |

As can be seen in Table 3 above, when the skin was treated with the paper mulberry extract of Preparation Example 1, transepidermal water loss was restored to normal level, and barrier impairment was restored.

### Reference Example 1

### Preparation of Formulations of Example 2 and Comparative Example 2

Using the paper mulberry extract obtained in Preparation Example 1, nourishing cream formulations of Example 2 and Comparative Example 2 were prepared according to the components and contents in Table 4 below. The amounts in Table 4 are by weight %.

TABLE 4

| | | | Comparative |
|---|---|---|---|
| Component | Example 2 | Example 3 | Example 2 |
| Purified water | To 100 | To 100 | To 100 |
| Paper mulberry extract | 0.5 | 0.1 | — |
| (Preparation Example 1) | | | |
| Vegetable hydrogenated | 1.50 | 1.50 | 1.50 |
| oil | | | |
| Stearic acid | 0.60 | 0.60 | 0.60 |
| Glycerol stearate | 1.00 | 1.00 | 1.00 |
| Stearyl alcohol | 2.00 | 2.00 | 2.00 |
| polyglyceryl-10 | 1.00 | 1.00 | 1.00 |
| pentastearate & behenyl | | | |
| alcohol & sodium | | | |
| stearoyl lactylate | | | |
| Arachidyl behenyl | 1.00 | 1.00 | 1.00 |
| alcohol & arachidyl | | | |
| glucoside | | | |
| Cetearyl alcohol & | 2.00 | 2.00 | 2.00 |
| cetearyl glucoside | | | |
| PEG-100 stearate & | 1.50 | 1.50 | 1.50 |
| glycerol oleate & | | | |
| propylene glycol | | | |
| Caprylic/capric | 11.00 | 11.00 | 11.00 |
| triglyceride | | | |
| Cyclomethicone | 6.00 | 6.00 | 6.00 |
| Preservative, fragrance | q.s. | q.s. | q.s. |
| Triethanolamine | 0.1 | 0.1 | 0.1 |

### Test Example 3

### Measurement of Effect on Increase in Skin Moisturization

In order to the effect of the paper mulberry extract on an increase in skin moisturization, the effect of improving skin moisturization was examined using the formulations of Example 2 and Comparative Example 2 of Table 4 above in the following manner.

Sixty 40-50-year-old men and women were divided into two groups (Example 2 and Comparative Example 2, respectively), each consisting of 30 persons. Each of the nourishing cream formulations was applied to the face twice a day for 4 weeks. Before application, 1 week, 2 weeks and 4 weeks after application, and 2 weeks after stop of application (6 weeks after application), the skin water content was measured using Corneometer CM825 (C+K Electronic Co., Germany) under the conditions of constant temperature and constant humidity (24° C., 40% relative humidity). The results of the measurement are shown in Table 5 below. The results in Table 5 are expressed as percentages of increases in skin water content after treatment relative to skin water content immediately before treatment.

TABLE 5

| | Increase (%) in water content | | | |
|---|---|---|---|---|
| | After | After | After | After |
| Test group | 1 week | 2 weeks | 4 weeks | 6 weeks |
| Example 2 | 33 | 40 | 42 | 34 |
| Comparative Example 2 | 30 | 32 | 32 | 15 |

As can be seen from the results in Table 5 above, when the cream of Comparative Example 2 was applied to the skin, an increase in water content of about 30% up to 4 weeks after application was shown, but the skin water content decreased after stop of application. However, in the case wherein the cream of Example 2 containing the paper mulberry extract was applied, an increase in skin water content of 30% or more was shown even after stop of application. This suggests that the inventive composition containing the paper mulberry extract has an excellent skin-moisturizing effect.

### Test Example 4

### Measurement of Effect on Inhibition of Elastase Activity

The elastase inhibitory activity of the paper mulberry extract prepared in Preparation Example 1 was measured in comparison with that of EGCG. The elastase and substrate used were commercially purchased from Sigma Aldrich (Cat. No. E0127, USA).

The elastase inhibitory activity was measured in the following manner.

In a 96-well plate, 50 μL of a solution of the extract of Preparation Example 1 in 10 mg/L Tris-HCL buffer (pH 8.0) was mixed with 50 μL of 20 μg/mL elastase type III solution. 250 μM of EGCG was used as a positive control, and distilled water was used as a negative control. Then, 100 μL of 0.4514 mg/mL N-SUCCINYL-ALA-ALA-ALA-p-NITROANILIDE in the same buffer used above was added to the plate and allowed to react at 25° C. for 15 minutes. After completion of the reaction, the absorbance at 415 nm was measured. For correction, a blank test was carried out in the same manner.

The elastase inhibitory activity was calculated using the following equation. The results of the calculation are shown in Table 6 below.

Elastase activity inhibition(%)={1−(C−D)/(A−B)}×100

wherein

A: absorbance at 415 nm in the case in which the test sample was not added and the enzyme was added;

B: absorbance at 415 nm in the case in which the test sample was not added and the enzyme was not added;

C: absorbance at 415 nm in the case in which the test sample was added and the enzyme was added; and

D: absorbance at 415 nm in the case in which the test sample was added and the enzyme was not added.

TABLE 6

| Test material | Expression (%) |
|---|---|
| Negative control (untreated) | 100 |
| EGCG | 90 ± 1.2 |
| Paper mulberry extract | 85 ± 3.2 |
| (Preparation Example 1) | |

As can be seen in Table 6 above, the elastase inhibitory activity of the paper mulberry extract was similar to or higher than that of EGCG known as an elastase activity inhibitor, suggesting that the paper mulberry extract of the present invention has an excellent effect of inhibiting elastase activity.

### Test Example 5

### Collagenase (MMP-1) Inhibitory Ability

The collagenase production inhibitory ability of the paper mulberry extract prepared in Preparation Example 1 was measured in comparison with that of retinoic acid.

Human fibroblasts were added to a 96-well microtiter plate containing 2.5% fetal bovine serum-containing DMEM (Dulbecco's Modified Eagle's Media) at a density of 5,000 cells/well and were cultured in a 5% CO₂ incubate at 37° C. to a confluence of about 70-80%. Then, the cell culture was treated with 10 μg/ml of the paper mulberry extract of Preparation Example 1 for 24 hours and collected.

The production of collagenase in the collected cell culture was measured using a commercially available collagenase measurement instrument (Catalog #: RPN 2610, Amersham Pharmacia, USA). First, the collected cell culture was added to a 96-well plate to which primary collagenase antibody had been uniformly applied, and then the culture was subjected to an antigen-antibody reaction in an incubator for 3 hours. After 3 hours, chromophore-conjugated secondary collagen antibody was added to the 96-well plate and allowed to react for 15 minutes. After 15 minutes, a color developing agent (3,3′,5,5′-tetramethylbenzidine, Sigma) was added to the plate, and color development was induced for 15 minutes at room temperature. Then, 1M sulfuric acid was added to the plate to stop the color development reaction, and at the same time, the reaction solution was yellow in color. The degree of yellow varied depending on the degree of progression of the reaction.

The absorbance of the yellowish 96-well plate at 405 nm was measured using a spectrophotometer, and based on the measurements, the degree of synthesis of collagenase was calculated using the following equation 1. The results of the calculation are shown in Table 7 below. Herein, the absorbance of a cell culture collected from a group not treated with the test sample was used as a control.

Collagenase expression(%)=(absorbance of cell group treated with material/absorbance of control)×100 Equation 1

TABLE 7

| Test material | Expression (%) |
|---|---|
| Control | 100 |
| Retinoic acid | 75 ± 3.4 |
| Paper mulberry extract | 72 ± 2.1 |
| (Preparation Example 1) | |

As can be seen in Table 7 above, the expression of collagenase in the cells treated with the paper mulberry extract was similar to that in the cells treated with retinoic acid known as a collagenase inhibitor, suggesting that the collagenase expression inhibitory effect of the paper mulberry extract was similar to that of retinoic acid.

The above results indicate that the paper mulberry extract according to the present invention has an effect of inhibiting matrix metalloproteinase (MMP-1).

### Test Example 6

### Examination of Effect on Improvement in Skin Elasticity

In order to measure the effect of the paper mulberry extract on the improvement in skin elasticity, the effect in the improvement in skin elasticity was evaluated using the formulations of Example 3 and Comparative Example 2 of Table 4 in the following manner.

Forty 30-40-year-old healthy women were divided into two groups (for Example 3 and Comparative Example 2, respectively), each consisting of 20 persons. Each of the nourishing cream formulations was applied to the face once a day for 12 weeks, and then the skin elasticity was measured using Cutometer SEM 575 (C+K Electronic Co., Germany). The results of the measurement are shown in Table 8 below. The results in Table 8 are expressed as ΔR8 (R8 (left)-R8 (right)) values in which the R8 values indicate viscoelasticity.

TABLE 8

| Test material | Skin elasticity effect |
|---|---|
| Example 3 | 0.42 |
| Comparative Example 2 | 0.10 |

As can be seen in Table 8, the formulation of Example 3 containing the paper mulberry extract of the present invention showed increased skin elasticity compared to the formulation of Comparative Example 2.

This suggests that a cosmetic composition containing the paper mulberry extract of the present invention is very effective in improving skin elasticity.

### Test Example 7

### Evaluation of Effect on Reduction in Skin Wrinkles

In order to measure the effect of the paper mulberry extract on a reduction in skin wrinkles, the effect on a reduction in wrinkles was examined using the formulations of Example 3 and Comparative Example 2 of Table 4 in the following manner.

Forty healthy women in their 40's were divided into two groups (for Example 3 and Comparative Example 2, respectively), each consisting of 20 persons. Each of the cream formulations was applied to the face of each person once a day for 12 weeks, and then skin replicas were obtained by applying silicon rubber to the skin. The replicas were photographed, and wrinkles were analyzed by Visiometer SV600 (Courage+Khazaka electronic GmbH, Germany). The results of the analysis are shown in Table 9 below. The results in Table 9 are expressed as the averages of values obtained by subtracting parameter values before application from parameter values after 12 weeks of application.

TABLE 9

| | Results after 8 weeks of application | | | | |
|---|---|---|---|---|---|
| | R1 | R2 | R3 | R4 | R5 |
| Example 3 | −0.19 | −0.18 | −0.10 | −0.03 | −0.03 |
| Comparative | 0.27 | 0.26 | 0.21 | 0.03 | 0.03 |
| Example 2 | | | | | |

R1: difference between the highest value and the lowest value of the wrinkle contour line;
R2: average of five R1 values of the wrinkle contour line randomly divided into 5 portions;
R3: the highest value of the five R1 values;
R4: average of the difference between the peak and the valley on the baseline of the wrinkle contour line; and
R5: difference between the baseline of the wrinkle contour line and the wrinkle contour.

As can be seen in Table 9, the skin external composition of Example 2 has a very excellent effect of reducing skin wrinkles.

### Reference Example 2

### Preparation of Formulations of Example 4 and Comparative Examples 3 and 4

Using the paper mulberry extract obtained in Preparation Example 1, external use formulations of Example 4 and Comparative Examples 3 and 4 were prepared according to the components and contents in Table 10 below. The contents in Table 10 are by wt %.

Specifically, the formulation of Example 4 contains the paper mulberry extract of Preparation Example 1, the formulation of Comparative Example 3 does not contain a component effective in alleviating acne, and the formulation of Comparative Example 4 contains erythromycin which is a standard for antimicrobial activity and has been frequently used as an acne-treating agent.

The formulations of Example 4 and Comparative Examples 3 and 4 were prepared in the following manner. The components of phase A in Table 10 were completely dissolved, and components of phase B were completely dissolved in a separate container. Then, phase B was added to and mixed with phase A. Components of phase C were added to the mixture in the amounts shown in Table 10 and were uniformly mixed. Then, the mixture was filtered, thereby preparing formulations.

TABLE 10

| | | | Comparative | Comparative |
|---|---|---|---|---|
| | | Example 4 | Example 3 | Examples 4 |
| A | Deionized Water | To 100 | To 100 | To 100 |
| | EDTA—2Na | 0.02 | 0.02 | 0.02 |
| | Glycerin | 5.0 | 5.0 | 5.0 |
| B | Ethanol | To 100 | To 100 | To 100 |
| | PEG-60 hydrogenated | 0.4 | 0.4 | 0.4 |
| | castor oil | | | |
| | Perfume | 0.04 | 0.04 | 0.04 |
| C | Paper mulberry | 5.0 | — | — |
| | extract (Preparation | | | |
| | Example 1) | | | |
| | erythromycin | — | — | 5.0 |

### Test Example 8

### Test for Antimicrobial Activity Against Propionibacterium acnes

Using the cosmetic compositions of Example 4 and Comparative Examples 3 and 4, antimicrobial activity against Propionibacrium acnes (ATCC 6919: medium-BHI broth) that causes acne was tested.

Antimicrobial activity against Propionibacterium acnes was tested in the following manner.

(1) Preparation of Microbial Test Solution

A culture obtained by inoculating Propionibacterium acnes into BHI broth and anaerobically culturing the Propionibacterium acnes was used.

(2) Preparation of Diluted Solution

A diluted solution was obtained by mixing 0.15 ml of the microbial test solution with 15 ml of BHI broth (pH 6.8) or LB broth (pH 4.5).

(3) Preparation of Samples

Cosmetic compositions of Example 3 and Comparative Examples 3 and 4 were used as samples without dilution.

(4) Test for Antimicrobial Activity

1) Each sample is added to line 1 of a 96-well plate according to starting concentration and the diluted solution is added thereto to a total volume of 200 μl.

2) The mixture of line 1 is well mixed and 100 μl is taken and added to line 2 and mixed well, and then 100 μl is taken and added to line 3. In this manner, double dilution is performed.

3) The mixture is stationary-incubated at 32° C. for 24 hours and 48 hours, and whether the microorganisms multiply determined based on the degree of suspension, and the minimum concentration at which the microorganisms do not multiply is determined to be MIC (minimum inhibitory concentration). If the mixture is not clear to make it difficult to determine whether the microorganisms multiply, microscopic observation is performed.

The results of the test for antibacterial activity against Propionibacterium acnes are shown in Table 11 below. MIC is expressed in terms of the concentration of the active ingredient in the formulation.

TABLE 11

| | pH | Propionibacterium acnes |
|---|---|---|
| Example 4 | 5.7 | <87 ppm |
| Comparative | 5.7 | >maximum concentration (no |
| Example 3 | | antimicrobial activity) |
| Comparative | 5.7 | <100 ppm |
| Example 4 | | |

In MIC, the lower the ppm concentration, the higher the antimicrobial activity against Propionibacterlum acnes. Thus, it can be seen that the formulation of Example 4 shows a ppm concentration lower than that of the formulation of Comparative Example 4 containing erythromycin (known acne therapeutic agent), suggesting that it has excellent antimicrobial activity against Propionibacterium acnes.

### Test Example 9

### Test for Inhibition of Lipogenesis

Mouse fibroblast 3T3-L1 cells were seeded into a 6-well culture plate containing 10% fetal bovine serum (FBS)-containing DMEM (Dulbeco's modified eagle's medium (GIBCO BRL, Life Technologes) at a density of 1×10⁵ cells/well. After 2 days, the medium was replaced with fresh DMEM (containing 10% FBS), followed by culture for 2 days. Then, the cultured cells were treated with DMEM (containing 10% FBS) containing 1 μg/ml insulin, 0.5 mM IBMX and 0.25 μM dexamethasone to induce differentiation, and after 2 days, the medium was replaced with insulin-containing DMEM, followed by incubation for 5 days. After 5 days, the medium was replaced with normal medium (DMEM containing 10% FBS), and the cells were incubated until the cells morphologically differentiated into adipocytes.

In order to measure the effect of the paper mulberry extract on the inhibition of the accumulation of fat in adipocytes, the differentiated 3T3-L1 adipocytes were subjected to Sudan III staining (S4136, Sigma-Aldrich). The adipocytes were fixed with 4% paraformaldehyde (pH 7.2) in phosphate buffer at room temperature, and then washed with PBS (phosphate buffered saline), after which the cells were stained with Sudan III and photographed for visual comparison. As a control, medium (not treated with the test material or the positive control) was used, and as the positive control, 50 μM caffeine was used. The degree of inhibition of fat accumulation was expressed as +++, ++ and + depending on the degree of staining. The results are shown in Table 12.

TABLE 12

| Sample | Inhibition (%) |
|---|---|
| Control | +++ |
| Positive control | + |
| Paper mulberry extract | + |
| (Preparation Example 1) | |

As can be seen in FIG. 12, the paper mulberry extract according to the present invention has the effect of inhibiting lipogenesis. Thus, it can inhibit the occurrence of acne by reducing sebum through the inhibition of lipogenesis.

### Test Example 10

### Test for Alleviation of Acne, Reduction of Serum Secretion, and Irritation

12 persons having acne were allowed to use the cosmetic compositions of Example 4 and Comparative Examples 3 and 4 for one month. The alleviation of acne was evaluated on a five-point scale: 1=not effective; 3=moderately effective; 5=very effective. The test results are shown in Table 13 as the averages of values for 12 persons.

The timing of the disappearance of acne was based on the date when the disappearance was observed, and whether acne recurred was based on the results after 1 months. The reduction in sebum secretion was evaluated on a five-point scale: 1=not effective; 3=moderately effective; 5=very effective. The test results are shown in Table 13 as the averages of values for 12 persons. Skin irritation was expressed as (number of subjects showing irritation)/(total number of subjects).

TABLE 13

| | Alleviation | Timing of | | | |
|---|---|---|---|---|---|
| | of | disappearance | Recur- | Reduction | |
| | inflammatory | of acne | rence | in sebum | Irri- |
| | acne | comedonica | of acne | secretion | tation |
| Example 4 | 3.2 | 4 days | Absent | 4.5 | 0/12 |
| Comparative | 2.1 | 13 days | Present | 2.0 | 0/12 |
| Example 3 | | | | | |
| Comparative | 4.2 | 2 days | Absent | 4.1 | 9/12 |
| Example 4 | | | | | |

As can be seen in Table 13 above, the composition of Example 4 did not show the recurrence of acne, unlike the composition of Comparative Example 3, and had an excellent effect on the alleviation of acne. Meanwhile, the composition of Comparative Example 4 containing the standard substance for antimicrobial activity showed antimicrobial activity similar to that of Example 4, but showed strong irritation during use, suggesting that it can cause skin irritation when it is used for a long period of time.

### Test Example 11

### Effect on Alleviation of Inflammation

Anti-inflammatory effect was evaluated based on the inhibition of prostaglandin production. The anti-inflammatory effect of the paper mulberry extract of Preparation Example 1 was measured using macrophages. First, to microphages collected from the abdominal cavity of mice, aspirin was added to a final concentration of 500 M so as to irreversibly inhibit the activity of cyclooxygenase (COX) in the cells. Then, 100 μl of the cell suspension was added to each well of a 96-well plate and cultured in a 5% CO₂ incubator at 37° C. for 2 hours so that the macrophages were attached to the plate surface. Then, the attached macrophages were washed three times with PBS and used to test the effect of the extract. The cultured macrophages were added to 1%(w/v) LPS-containing RPMI medium at a concentration of 5×10⁴ cells/ml and cultured for 12 hours to induce the production of prostaglandin, after which they were treated with 100 μl of the extract. Released prostaglandin was quantitatively analyzed by ELISA.

Herein, the prostaglandin production inhibitory activity of the extract was expressed as the percentage of prostaglandin production in the group treated with LPS together with the sample relative to 100% for the difference in prostaglandin production between the LPS-treated group and the non-LPS-treated group. The results (inhibitory effect on prostaglandin production) are shown in Table 14 below.

TABLE 14

| | |
|---|---|
| Blank | 100% |
| Control (treated with aspirin) | 25.0% |
| Paper mulberry extract | 26.1% |
| (Preparation Example 1) | |

As can be seen in Table 14 above, treatment with the paper mulberry extract showed a very high effect on the inhibition of prostaglandin production, like the control treated with aspirin.

This suggests that the paper mulberry extract of the present invention shows an excellent effect on the alleviation of inflammation.

### Test Example 12

### Effect on Inhibition of 5α-Reductase

In order to examine the effect of inhibiting 5α-reductase activity, the ratio of [¹⁴C]testosterone-to-[14^{C}]dihydrotestosteron conversion in HEK293-5αR2 cells was measured. HEK293 cells transfected with p3×FLAG-CMV-5αR2 were added to a 24-well plate at a density of 2.5×10⁵ cells/well and cultured (Park et al., 2003, JDS. Vol. 31, ppl 91-98). The next day, the medium was replaced with fresh medium containing an enzyme substrate and an inhibitor. As the substrate of the medium, 0.05 μCi [¹⁴C]testosterone (Amersham Pharmacia biotech, UK) was used. In order to measure the degree of inhibition, 10 μg/ml of the paper mulberry extract of Preparation Example 2 was added to the cells which were then incubated in a 5% CO₂ incubator at 37° C. for 2 hours. As a positive control, finasteride was used. The culture medium was collected and, steroid was extracted with 800 μl of ethyl acetate. The upper organic solvent layer was separated and dried, and the remaining material was dissolved in 50 μl of ethyl acetate and developed on silica plastic sheet kieselgel 60 F254 using ethyl acetate-hexane (1:1) as a developing solvent.

The plastic sample was dried in air, and the amount of isotopes was measured using a Vas system. Specifically, the dried plastic sheet together with an X-ray film was placed in a Vas cassette, and after 1 week, the amounts of the testosterone and dihydrotestosterone isotopes were measured. The results of the measurement are shown in Table 15 below.

TABLE 15

| Sample | Conversion (%) | Inhibition (%) |
|---|---|---|
| Paper mulberry extract | 32.1 | 33.1 |
| Control | 48.0 | — |
| Positive control (finasteride) | 27.6 | 42.5 |

(1) conversion: radioactivity in the DHT region/total radioactivity
(2) inhibition: 100 × (conversion of control − conversion of sample)/conversion of control

As can be seen from the results in Table 15 above, the paper mulberry extract of the present invention blocks the conversion of testosterone to dihydrotestosterone by effectively inhibiting the activity of 5α-reductase that converts testosterone to dihydrotestosterone to enter the nucleus by binding to receptor protein in the cytoplasm so as to activate sebaceous gland cells and stimulate the differentiation of the cells to induce excessive secretion of sebum from sebaceous glands.

This suggests that the paper mulberry extract of the present invention has an excellent effect on the inhibition of 5α-reductase, and thus is effective in inhibiting excessive secretion of sebum.

### Reference Example 3

### Preparation of Formulations of Example 4 and Comparative Example 5

Using the paper mulberry extract of Preparation Example 2, lotion formulations of Example 4 and Comparative Example 5 were prepared according to the components and contents shown in Table 16 below. The contents in Table 16 are by wt %.

TABLE 16

| | | Comparative |
|---|---|---|
| Component | Example 5 | Example 5 |
| 1. cetearyl alcohol | 1.0 | 1.0 |
| 2. lipophilic glyceryl stearate | 1.0 | 1.0 |
| 3. glyceryl stearate SE | 1.5 | 1.5 |
| 4. phytosqualane | 3 | 3 |
| 5. hydrogenated polydecene | 2 | 2 |
| 6. dimethicone | 0.5 | 0.5 |
| 7. polysorbate 60 | 1 | 1 |
| 8. sorbitan sesquioleate | 0.4 | 0.4 |
| 9. methylparaben | 0.1 | 0.1 |
| 10. propylparaben | 0.05 | 0.05 |
| 11. purified water | To 100 | To 100 |
| 12. butylene glycol | 5 | 5 |
| 13. polyacrylate-13* polyisobutene | 0.5 | 0.5 |
| * polysorbate 20 | | |
| 14. paper mulberry extract | 1 | — |

Method for Preparation of Formulations of Example 5 and Comparative Example 5

1) Components 11 to 14 were uniformly mixed with each other while they were heated to 70° C., thereby preparing an aqueous phase.

2) Components 1 to 10 were uniformly mixed with each other while they were heated to 70° C., thereby preparing an oil phase.

3) The oil phase of 2) was added to the aqueous phase 1) and homomixed at 7,200 rpm for 6 minutes.

4) The mixture of 3) was cooled to room temperature.

### Test Example 13

### Effect on Inhibition of Sebum Secretion

In order to measure the effect of the paper mulberry extract on the inhibition of sebum secretion, the effect on the inhibition of sebum secretion was evaluated using the formulations of Example 5 and Comparative Example 5 of Table 16 in the following manner.

10 men and women in which a large amount of sebum was secreted were selected, and the lotions of Example 5 and Comparative Example 5 were applied to the appointed areas every day for 4 weeks. To determine the effect on sebum reduction, The amount of sebum was measured using a sebumeter, and the results of the measurement are shown in Table 17 below.

TABLE 17
Effect on inhibition of sebum secretion

| | Average reduction (%) in | | Average reduction (%) in | |
|---|---|---|---|---|
| | sebum after 2 weeks | | sebum after 4 weeks | |
| | | Comparative | | Comparative |
| | Example 5 | Example 5 | Example 5 | Example 5 |
| Average | 17.2 ± 3.4 | 5.1 ± 2.5 | 18.3 ± 4.2 | 6.8 ± 3.3 |
| value | | | | |

As can be seen from the results in Table 17 above, the formulation of Example 5 containing the paper mulberry extract of the present invention as an active ingredient effectively inhibited excessive secretion of sebum compared to the formulation of Comparative Example 5 which does not contain the paper mulberry extract.

This suggests that a skin external composition containing the paper mulberry extract of the present invention has an excellent effect of inhibiting sebum secretion.

### Test Example 14

### Inhibitory Effect on Production of Reactive Oxygen Species

Keratinocytes isolated from human epidermal tissue were seeded into a 24-well cell culture plate at a density of 5×10⁴ cells per well and cultured for 24 hours. After 16 hours, the cells were treated with the paper mulberry extract of Preparation Example 2 at a concentration of 1%. After 2 hours, the culture medium was removed, and 100 μl of phosphate buffered saline (PBS) was added to each well. The keratinocytes were irradiated with 30 mJ/cm² of UV light using a UV B lamp (Model: F15T8, UV B 15 W, Sankyo Dennki, Japan), after which PBS was removed and 200 μl of the keratinocyte culture was added to each well. The cells were treated with the paper mulberry extract, and the amount of reactive oxygen species (ROS) that increased by UV irradiation was measured at varying points of time. The amount of ROS was measured with reference to the method of Tan that measure the fluorescence of DCF-DA (dichlorofluorescein diacetate) oxidized by ROS (Tan et al., 1998, J. Cell Biol. Vol. 141, pp 1423-1432). The results of the measurement are shown in Table 18. The results in Table 18 are expressed as percentages relative to the ROS of the control.

TABLE 18

| | Time after irradiation of | | |
|---|---|---|---|
| | 30 mJ/cm² of UVB | | |
| | 0 hr | 2 hr | 3 hr |
| Vehicle | 100 | 244 | 287 |
| UVB + vehicle | 100 | 325 | 381 |
| UVB + paper mulberry extract | 100 | 273 | 301 |
| (Preparation Example 2) | | | |

As can be seen in Table 18 above, the paper mulberry extract of the present invention has an excellent antioxidant effect of effectively inhibiting the production of ROS known to cause skin damage by UV rays.

This suggests that the paper mulberry extract of the present invention can prevent the enlargement of pores by inhibiting oxidation and preventing aging and protect the skin from stimuli.

### Test Example 15

### Stimulation of Collagen Biosynthesis

The effect of the paper mulberry extract on collagen biosynthesis was measured in comparison with TGF-beta.

First, fibroblasts were seeded into a 24-well plate at a density of 10⁵ cells/well and cultured in serum-free DMEM medium for 24 hours to a confluence of about 90%. Then, the cells were treated with each of a solution of the paper mulberry extract of the present invention and 10 ng/ml of TGF-beta dissolved in serum-free medium and incubated in a CO₂ incubator for 24 hours. The supernatants of the cell cultures were collected and the amount of procollagen was measured using a procollagen type (I) ELISA kit. The results of the measurement are shown in Table 19. The values of collagen synthesis (%) in Table 19 are expressed as percentages relative to 100 for the control.

TABLE 19

| Sample | Collagen synthesis (%) |
|---|---|
| Control | 100 |
| TGF-beta | 183.5 ± 13.1 |
| Paper mulberry extract | 142.1 ± 5.2 |

As can be seen from the results in Table 19 above, the paper mulberry extract of the present invention showed high ability to synthesize collagen, like the positive control TGF-beta.

This suggests that the paper mulberry extract of the present invention can reduce pore size by increasing the production of collagen around pores.

### Test Example 16

### Test for Effect on Pore Size Reduction

In order to measure the effect of the paper mulberry extract on pore size reduction, the effect on pore size reduction was evaluated using the formulations of Example 5 and Comparative Example 5 of Table 16 in the following manner.

10 men and women having large pore size were selected, and the lotions of Example 5 and Comparative Example 5 were applied to the face every day for 4 weeks. To determine the effect on pore size reduction, photographs were taken before application and after 4 weeks of application and visually evaluated by experts. The evaluation was made on a six-point scale (0 to 5; 0: not reduced; 5: very reduced), and the results of the evaluation are shown in Table 20 below.

TABLE 20

| Test material | Score |
|---|---|
| Example 5 | 3.2 |
| Comparative Example 5 | 0.8 |

As can be seen from the results in Table 20 above, the paper mulberry extract of the present invention has an excellent effect of reducing pore size.

### Test Example 17

### Effect on Reduction in Expression of Skin Inflammatory Factor

In order to measure the effect of the paper mulberry extract of the present invention on the inhibition of the expression of the skin inflammatory factor PGE-2, ELISA (Enzyme Linked ImmunoSorbent Assay) was carried out (SE Dunsmore, et al., J Biol Chem, 271: 24576-24582, 1996).

Cells were cultured in medium containing yellow dust (0.1 ppm) for 24 hours, and the medium was replaced with fresh medium. The cells were treated with the paper mulberry extract and cultured for 24 hours. Then, the culture medium was collected, and the cells were coated on a 96-well plate. Primary antibody (monoclonal antibody) was added to the plate and allowed to react at 37° C. for 90 minutes. Then, the cells were allowed to react with the secondary antibody alkaline phosphatase-conjugated anti-mouse IgG for about 90 minutes, washed with buffer solution, and then allowed to react with alkaline phosphatase substrate solution (1 mg/ml p-nitrophenyl phosphate in diethanolamine buffer) at room temperature for 30 minutes, and the absorbance at 405 nm was measured using a spectrophotometer. As a control, a cell culture not treated with the paper mulberry extract of the present invention was used. The inhibition of PGE-2 expression was calculated using the following equation 2, and the results of the calculation are shown in Table 21 below.

Inhibition(%) of PGE-2 expression=(A−B)/A×100 Equation 2

wherein

A: absorbance of well containing no sample;

B: absorbance of well containing sample.

TABLE 21

| Test material | PGE-2 expression inhibition (%) |
|---|---|
| Control | — |
| Paper mulberry extract | 26.1 |
| (Preparation Example 2) | |

As can be seen in Table 21 above, the paper mulberry extract of the present invention effectively inhibits the expression of the skin inflammatory factor PGE-2.

This suggests that the paper mulberry extract of the present invention has an excellent effect of preventing skin trouble by inhibiting the expression of the skin inflammatory factor.

### Test Example 18

### Effect on Complexion Improvement

In order to measure the effect of the paper mulberry extract on complexion improvement, the effect on blood circulation stimulation was evaluated using the formulations of Example 5 and Comparative Example 5 of Table 16 in the following manner.

The effect on the stimulation of skin blood circulation was evaluated by measuring the degree of blood circulation in the skin using LDPI (Laser Doppler Perfusion Imager). LDPI is widely known as a device for measuring blood circulation in the skin and is a very sensitive device capable of measuring not only the velocity and amount of blood in the capillary vessel of the skin, but also blood flow in arterioles and venules.

In a constant-temperature and constant-humidity chamber, the face was washed with soap and adapted for 30 minutes, and initial values were measured using LDPI. 20 women whose hands and feet were usually cold participated in the test, and the initial blood flow rate in the portion below the forehead of the participants was measured using LDPI.

The formulations of Example 5 and Comparative Example 5 were applied to the subjects for one week, and then the blood flow rate and the skin temperature were compared with the initial measurement values, and the results of the comparison are shown in Table 22 below.

TABLE 22

| | Example 5 | Comparative Example 5 |
|---|---|---|
| Change (%) after one | 16.3% | 1.9% |
| week of application | | |

As can be seen from the results in Table 22 above, the formulation of Example 5 containing the paper mulberry extract of the present invention improves complexion by more effectively stimulating blood circulation as compared to the formulation of Comparative Example 5 which does not contain the paper mulberry extract.

This ultimately suggests that a composition containing the paper mulberry extract of the present invention can contribute to the effective transfer of nutrients to the skin and the inhibition and delay of aging.

### Test Example 19

### Effect on Improvement in Skin Tone

In order to measure the effect of the paper mulberry extract on skin tone improvement, the effect on skin tone improvement was evaluated using the formulations of Example 5 and Comparative Example 5 of Table 16 in the following manner.

Each of the formulations of Example 5 and Comparative Example 5 was applied to 10 subjects in the evening once a day for one week, and then the degree of skin tone improvement was evaluated using Facial Stage DM-3 (Moritex, Japan). The degree of skin tone improvement was determined based on the changes in the brightness and saturation values of the skin. The results are shown in Table 23 below.

TABLE 23

| | Example 5 | Comparative Example 5 |
|---|---|---|
| Skin tone | Brightness (mean ± SD) | Brightness (mean ± SD) |
| improvement | 2.1 ± 0.3 | 0.8 ± 0.4 |
| (%) | Saturation (mean ± SD) | Saturation (mean ± SD) |
| | 1.5 ± 0.2 | 0.5 ± 0.4 |

As can be seen from the results in Table 23 above, the formulation of Comparative Example 5 which does not contain the paper mulberry extract of the present invention showed no significant effect on skin tone improvement, but the formulation of Example 5 containing the paper mulberry extract showed a significant improvement in skin tone after application compared to before application.

### Test Example 20

### Effect on Stimulation of Decomposition of Triglyceride in Adipocytes

Mouse fibroblast 3T3-L1 cells were seeded into a 6-well culture plate containing 10% fetal bovine serum (FBS)-containing DMEM (Dulbeco's modified eagle's medium, GIBCO BRL, Life Technologes) at a density of 1×10⁵ cells/well. After 2 days, the medium was replaced with fresh DMEM (containing 10% FBS), followed by culture for 2 days. Then, the cultured cells were treated with DMEM (containing 10% FBS) containing 1 μg/ml insulin, 0.5 mM IBMX and 0.25 μM dexamethasone to induce differentiation, and after 2 days, the medium was replaced with insulin-containing DMEM, followed by incubation for 5 days. After 5 days, the medium was replaced with normal medium (DMEM containing 10% FBS), and the cells were incubated until the cells morphologically differentiated into adipocytes.

In order to evaluate the effect of the paper mulberry extract on the stimulation of decomposition of triglyceride in adipocytes, a test was carried out using the differentiated 313-L1 adipocytes. The 3T3-L1 adipocytes were washed twice with PBS (phosphate buffered saline), and colorless DMEM containing 0.5% fatty acid-free bovine serum albumin (BSA) was added to the cells, and a fraction of the cells was taken and used in the test. As a control, a medium not treated with the test material or the positive control was used. A value of 100% for the control was used as a standard for comparison. In addition, 50 μM caffeine was used as the positive control. The degree of lypolysis was determined by measuring the level of glucose released from the adipocytes into the culture medium. To measure the level of glucose, the culture was subjected to a color development reaction using the GPO-trinder kit (Sigma, St. Louis, Mo., U.S.A.), and the absorbance at 540 nm was measured using an ELISA reader. The results are shown in Table 24 below.

TABLE 24

| | | | Paper mulberry extract |
|---|---|---|---|
| | Control | Caffeine | (Preparation Example 2) |
| Level of released | 100% | 115% | 140% |
| glycerol | | | |

As can be seen in Table 24 above, the level of glucose released from the adipocytes into the culture medium was significantly higher in the group treated with the paper mulberry extract of the present invention than in the control group. In addition, the group treated with treated with the paper mulberry extract of the present invention showed a significantly high lypolysis compared to the group treated with the positive control caffeine.

### Reference Example 4

### Preparation of Formulations of Example 6 and Comparative Example 6

Using the paper mulberry extract obtained in Preparation Example 2, lotion formulations of Example 6 and Comparative Example 6 were prepared according to the components and contents shown in Table 25 below. The contents in Table 25 are by wt %.

TABLE 25

| | Example | Comparative |
|---|---|---|
| Component | 6 | Example 6 |
| Purified water | To 100 | To 100 |
| Paper mulberry extract (Preparation | 1.0 | — |
| Example 2) | | |
| Vegetable hydrogenated oil | 1.50 | 1.50 |
| Stearic acid | 0.60 | 0.60 |
| polyglycerol-10 pentastearic & behenyl | 1.00 | 1.00 |
| alcohol & sodium stearoyl lactylate | | |
| Arachidyl behenyl alcohol & arachidyl | 1.00 | 1.00 |
| glucoside | | |
| Cetylaryl alcohol & cetearyl glucoside | 2.00 | 2.00 |
| PEG-100 stearate & glycerol oleate & | 1.50 | 1.50 |
| propylene glycol | | |
| Caprylic/capric triglyceride | 4.00 | 4.00 |
| Meadowfoam seed oil | 3.00 | 3.00 |
| Cetyl octanoate | 4.00 | 4.00 |
| Cyclomethicone | 6.00 | 6.00 |
| Methyl paraben | 0.20 | 0.20 |
| Propyl paraben | 0.10 | 0.10 |
| Disodium EDTA | 0.02 | 0.02 |
| Trimethanolamine | 0.13 | 0.13 |
| Glycerin | 8.00 | 8.00 |
| Carbomer | 0.13 | 0.13 |

### Test Example 21

### Slimming Effect

The slimming effect of the paper mulberry extract was measured using the formulations of Example 6 and Comparative Example 6 of Table 25 in the following manner.

On forty 25-46-year-old women having regional obesity or cellulite and a BMI (Body Mass Index, weight (kg)/height (m)²) of 21-27, the formulations of Example 6 and Comparative Example 6 were applied to one thigh with massage at home twice (morning and evening) a day for 4 weeks. The effects of the formulations were analyzed by 8-week instrumental evaluation, researcher (dermatologist) evaluation and questionnaire evaluation.

Ultrasonic measurement of the subcutaneous fat layer thickness (unit: mm) was performed using the Ultrasound-EuB 415 US scanner, and the obtained values were compared between before and after application using Student-t test or Wilcoxon test to analyze statistical significance (significant level α=0.05). The results are shown in Table 26 below.

TABLE 26

| | Example | Comparative |
|---|---|---|
| | 6 | Example 6 |
| Reduction (%) in | 26.4% | 9.1% |
| subcutaneous fat thickness | | |

As can be seen in Table 26, the use of the formulation of Example 6 containing the paper mulberry extract of the present invention showed a significant reduction in the circumference of the thigh compared to the formulation of Comparative Example 6 that does not contain the paper mulberry extract. During the test period, no change in the bodyweight of the subjects was observed.

In addition, the skin elasticity of the subjects was measured using Cutometer SEM 575 (C+K Electronic Co., Germany). The degree of cellulite was visually evaluated by professional researchers. The obtained values were compared between before and after application using Student-t test or Wilcoxon test to analyze statistical significance (significant level α=0.05). Among the evaluation indices, elasticity was evaluated based on the change in R2 value (closer to 1 is better) indicating gross elasticity, and the degree of cellulite was evaluated on a five-point scale (0-4; 0=very much cellulite; 4=no cellulite) by visual evaluation. The results of the evaluation are shown in Table 27 below.

TABLE 27

| | Example | Comparative |
|---|---|---|
| | 6 | Example 6 |
| Change in cellulite score | 1.7 | 0.4 |
| (score before application − score | | |
| after application) | | |
| Change in elasticity: ΔR2 (R2 | 0.368 | 0.099 |
| before application − R2 after | | |
| application) | | |

As can be seen in Table 27 above, the results of evaluation by researchers showed that cellulite was statistically significantly reduced in the area applied with Example 6 as compared to in the area applied with Comparative Example 6 and that skin elasticity was increased in the area the area applied with Example 6.

Thus, the skin external composition containing the paper mulberry extract according to the present invention showed an excellent slimming effect by effectively reducing subcutaneous fat and cellulite and also increasing skin elasticity.

### Test Example 22

### Effect of Paper Mulberry Extract on Stimulation of MITF Expression in Transformed Cells

To examine the gray preventing effect of the paper mulberry extract, the effect of the paper mulberry extract on the stimulation of MITF expression was examined as disclosed in Korean Patent Application No. 10-2007-0072182 (entitled “A method for screening a gray hair-preventing substance using a transformed cell line and leukoplakia mice and a composition for preventing gray hair containing the gray hair-preventing substance”). The Melan-a melanocyte cell line MITF-GLuc (accession number: KCLRF-BP-00162) transformed with the expression vector pMITF-GLuc was cultured in RPMI 1640 medium containing 10% fetal bovine serum (FBS), 100 unit/ml penicillin-streptomycin (Gibco), 0.1 μM TPA (Sigma) and 400 μg/ml G418 under the conditions of 37° C. and 10% CO₂. The positive control IBMX was purchased from Sigma and used at a concentration of 100 μM. The transformed melanin cells (melan-a) were dispensed into a 24-well microtiter plate at a density of 50,000 cells/well. The next day, the dispensed cells were treated with the paper mulberry extract (1000× stock) of Preparation Example 1 at final concentrations of 10 and 50 ppm, a negative control group was treated with 0.1% DMSO, and a positive control group was treated with 100 μM IBMX, after which the cells were incubated at a temperature of 37° C. for 3 days. After the incubation, to quantify the amount of GLuc, a small amount of the medium was taken, transferred to a measurement plate and allowed to react with a substrate. Specifically, a small amount of the medium was taken from the cell culture dish and transferred to a measurement plate, after which 1× GLuc assay working solution (NEB) was added to the medium at a ratio of 4:1, and the amount of light emitted at 470 nM was measured using a luminometer. The results of the test are shown in Table 28 below.

TABLE 28

| | Percentage (%) of gluciferase |
|---|---|
| | production relative to |
| | negative control |
| Negative control (DMSO) | 100 |
| Positive control (100 μM IBMX) | 180 |
| Paper mulberry extract | 120 |
| (Preparation Example 1) (10 ppm) | |
| Paper mulberry extract | 145 |
| (Preparation Example 1) (50 ppm) | |

As can be seen in Table 28 above, the paper mulberry extract stimulated the expression of MITF in the transformed melanocytes.

### Test Example 23

### Evaluation of Effects of Paper Mulberry Extract on Gray Hair Prevention and Black Hair Induction in Leukoplakia Mice in which the Occurrence of Gray Hair was Stimulated

Leukoplakia mice (C57bl/6-Mitf^{mi-vit}) were purchased from The Jackson Lab (USA) and used. The effect of the paper mulberry extract on gray hair prevention in the mice was tested in the following manner. The back of 12-week-old mice was depilated such that the depilated area was the same between the mice. From the day following the depilation day, gray hair-preventing substances were applied to the depilated area twice a day. As a vehicle for the gray hair-preventing substance, a mixture of EtOH:1,3-BG:DW=3:2:5 (volume ratio) was used. The vehicle was used as a negative control, the vehicle containing 50 mM IBMX was used as a positive control, and the vehicle containing 2.5% paper mulberry extract of Preparation Example 1 was used as a test sample. After about 3 weeks, the difference in gray hair prevention effect between the materials has been distinguished, newly grown hairs were collected and the amount of melanin in the hairs was measured using esperase (Novozyme). Specifically, esperase was dissolved in buffer (50 mM Tris-HCl, 5 mM DTT, pH 9.3) at a concentration of 1 NPU/ml to prepare a reaction buffer. 5 mg of the mouse hair was added to the 1 ml of the reaction buffer, and the mixture was allowed to react with stirring at 37° C. at 1,000 rpm for 13 hours, and then separated into hair and a reaction solution by momentary centrifugation. The reaction solution thus obtained was added to a 96-well plate, and the absorbance at 405 nm was measured, thereby measuring the amount of melanin in the reaction solution. As described above, the leukoplakia mouse model in which the occurrence of gray hair was stimulated was treated with each of the negative control, the positive control and the test sample, and the effects of the negative control, the positive control and the test sample were measured visually and by measuring the amount of melanin in the hair. The results of the measurement are shown in Table 29 below.

TABLE 29

| | Ratio (%) of amount of |
|---|---|
| | melanin in hair relative to |
| | negative control |
| Negative control | 100 |
| Positive control (IBMX) | 105.9 |
| Paper mulberry extract | 110.1 |
| (Preparation Example 1) | |

As can be seen in Table 29 above, the paper mulberry extract can stimulate the induction of black hair in the leukoplakia mouse model by inhibiting gray hair and increasing the amount of melanin in the hair.

### Formulation Example 1

### Milk Lotion

Milk lotion was prepared using the composition shown in Table 30 below according to a conventional method.

TABLE 30

| Component | Content (wt %) |
|---|---|
| Purified water | Balance |
| Glycerin | 8.0 |
| Butylene glycol | 4.0 |
| Hyaluronic acid extract | 5.0 |
| Beta-glucan | 7.0 |
| Carbomer | 0.1 |
| Paper mulberry extract | 0.05 |
| (Preparation Example 1) | |
| Caprylic/capric triglyceride | 8.0 |
| Squalane | 5.0 |
| Cetearyl glucoside | 1.5 |
| Sorbitan stearate | 0.4 |
| Cetearyl alcohol | 1.0 |
| Preservative | q.s. |
| Fragrance | q.s. |
| Pigment | q.s. |
| Triethanolamine | 0.1 |

### Formulation Example 2

### Nourishing Lotion

Nourishing lotion was prepared using the composition shown in Table 31 below according to a conventional method.

TABLE 31

| Component | Content (wt %) |
|---|---|
| Purified water | Balance |
| Glycerin | 3.0 |
| Butylene glycol | 3.0 |
| Liquid paraffin | 5.0 |
| Beta-glucan | 7.0 |
| Carbomer | 0.1 |
| Paper mulberry extract | 3.0 |
| (Preparation Example 1) | |
| Caprylic/capric triglyceride | 3.0 |
| Squalane | 5.0 |
| Cetearyl glucoside | 1.5 |
| Sorbitan stearate | 0.4 |
| Polysorbate 60 | 1.5 |
| Preservative | q.s. |
| Fragrance | q.s. |
| Pigment | q.s. |
| Triethanolamine | 0.1 |

### Formulation Example 3

### Nourishing Cream

Nourishing cream was prepared using the composition shown in Table 32 below according to a conventional method.

TABLE 32

| Component | Content (wt %) |
|---|---|
| Purified water | Balance |
| Glycerin | 3.0 |
| Butylene glycol | 3.0 |
| Liquid paraffin | 7.0 |
| Beta-glucan | 7.0 |
| Carbomer | 0.1 |
| Paper mulberry extract | 3.0 |
| (Preparation Example 1) | |
| Caprylic/capric triglyceride | 3.0 |
| Squalane | 5.0 |
| Cetearyl glucoside | 1.5 |
| Sorbitan stearate | 0.4 |
| Polysorbate 60 | 1.2 |
| Preservative | q.s. |
| Fragrance | q.s. |
| Pigment | q.s. |
| Triethanolamine | 0.1 |

### Formulation Example 4

### Pack

A pack was prepared using the composition shown in Table 33 below according to a conventional method.

TABLE 33

| Component | Content (wt %) |
|---|---|
| Purified water | Balance |
| Glycerin | 4.0 |
| Polyvinyl alcohol | 15.0 |
| Hyaluronic acid extract | 5.0 |
| Beta-glucan | 7.0 |
| Allantoin | 0.1 |
| Paper mulberry extract | 0.5 |
| (Preparation Example 1) | |
| Nonylphenyl ether | 0.4 |
| Polysorbate 60 | 1.2 |
| Preservative | q.s. |
| Fragrance | q.s. |
| Pigment | q.s. |
| Ethanol | 6.0 |

### Formulation Example 5

### Ointment

An ointment was prepared using the composition shown in Table 34 below according to a conventional method.

TABLE 34

| Component | Content (wt %) |
|---|---|
| Purified water | Balance |
| Glycerin | 8.0 |
| Butylene glycol | 4.0 |
| Liquid paraffin | 15.0 |
| Beta-glucan | 7.0 |
| Carbomer | 0.1 |
| Paper mulberry extract | 1.0 |
| (Preparation Example 1) | |
| Caprylic/capric triglyceride | 3.0 |
| Squalane | 1.0 |
| Cetearyl glucoside | 1.5 |
| Sorbitan stearate | 0.4 |
| Cetearyl alcohol | 1.0 |
| Preservative | q.s. |
| Fragrance | q.s. |
| Pigment | q.s. |
| Beeswax | 4.0 |

### Formulation Example 6

### Massage Cream

Massage cream was prepared using the composition shown in Table 35 below according to a conventional method.

TABLE 35

| Component | Content (wt %) |
|---|---|
| Purified water | Balance |
| Glycerin | 8.0 |
| Butylene glycol | 4.0 |
| Liquid paraffin | 45.0 |
| Beta-glucan | 7.0 |
| Carbomer | 0.1 |
| Paper mulberry extract | 1.0 |
| (Preparation Example 1) | |
| Caprylic/capric triglyceride | 3.0 |
| Beeswax | 4.0 |
| Cetearyl glucoside | 1.5 |
| Sorbitan sesquioleate | 0.9 |
| Vaseline | 3.0 |
| Preservative | q.s. |
| Fragrance | q.s. |
| Pigment | q.s. |
| Paraffin | 1.5 |

### Formulation Example 7

### Hair Shampoo

Hair shampoo was prepared using the composition shown in Table 36 below according to a conventional method.

TABLE 36

| Component | Content (wt %) |
|---|---|
| Sodium lauryl sulfate (30%) solution | 20.0 |
| Coconut oil fatty acid | 5.0 |
| diethanolamide | |
| Polyquarternium-10 | 0.3 |
| Propylene glycol | 2.0 |
| Paper mulberry extract (Preparation | 0.1 |
| Example 1) | |
| Piroctone olamine | 0.5 |
| Yellow No. 203 | q.s. |
| Para-oxybenzoic acid ester | 0.2 |
| Combined fragrance | q.s. |
| Citric acid | q.s. |
| Purified water | Balance |

### Formulation Example 8

### Hair Conditioner

Hair conditioner was prepared using the composition shown in Table 37 below according to a conventional method.

TABLE 37

| Component | Content (wt %) |
|---|---|
| Cetyltrimethylammonium chloride (29%) | 7.0 |
| Distearyldimethylammonium chloride (75%) | 4.0 |
| Cetostearyl alcohol | 3.5 |
| Polyoxyethylene stearylether | 1.0 |
| Liquid paraffin | 2.0 |
| Propylene glycol | 1.5 |
| Paper mulberry extract | 0.1 |
| (Preparation Example 1) | |
| Combined fragrance | q.s. |
| Citric acid | q.s. |
| Purified water | Balance |

### Formulation Example 9

### Scalp Hair Tonic

Scalp hair tonic was prepared using the composition shown in Table 38 below according to a conventional method.

TABLE 38

| Component | Content (wt %) |
|---|---|
| Menthol | 0.1 |
| D-panthenol | 0.6 |
| Salicylic acid | 0.05 |
| Glycerin | 1.0 |
| Polyoxyethylene hydrogenated | 0.8 |
| castor oil | |
| Tocopherol acetate | 0.03 |
| Combined fragrance | q.s. |
| Paper mulberry extract | 0.1 |
| (Preparation Example 1) | |
| Ethanol | 30.0 |
| Purified water | Balance |

### Formulation Example 10

### Scalp Essence

Scalp essence was prepared using the composition shown in Table 39 below according to a conventional method.

TABLE 39

| Component | Content (wt %) |
|---|---|
| Ethanol | 30.0 |
| Polysorbate 60 | 1.5 |
| Glycerin | 3.0 |
| Carboxyvinyl polymer | 0.1 |
| Triethanolamine | 0.2 |
| Paper mulberry extract (Preparation | 0.1 |
| Example 1) | |
| Preservative | q.s. |
| Fragrance and pigment | q.s. |
| Purified water | Balance |

1. A method for improving at least one skin condition selected from the group consisting of moisturization, anti-aging, elasticity improvement, wrinkle reduction, acne alleviation, pore size reduction, sebum control, slimming, gray hair prevention, and leukoplakia prevention, comprising topically applying an effective amount of a water extract of paper mulberry plant to the skin of a subject in need of thereof,
wherein the water extract is prepared by a process consisting of:
(a) adding stem, root, leave, flower, and fruit of paper mulberry plant into water to obtain a mixture;
(b) conducting an extraction by boiling the mixture of (a); and
(c) filtering the resulting boiled mixture of (b) to obtain a filtrate, standing the filtrate, and concentrating the filtrate by distillation under reduced pressure.

2. The method of claim 1, wherein the moisturization effect of the extract is an effect of enhancing skin barrier function or inhibiting keratinocyte differentiation.

3. The method of claim 1, wherein the acne alleviation effect of the extract is an effect of inhibiting Propionibacterium acnes or inflammation.

4. The method of claim 1, wherein the slimming effect of the extract is an effect of decomposing triglyceride or removing cellulite.

5. The method of claim 1, wherein the gray hair or leukoplakia prevention effect of the extract is an effect of stimulating expression of MITF or tyrosinase in melanocytes.
